(19) United States
(12) Patent Application Publication
(10) Pub. No.: US 2009/0053335 A1
(43) Pub. Date: Feb. 26, 2009

(54) HERBAL COMPOSITIONS FOR THE INHIBITION OF HUMAN IMMUNODEFICIENCY VIRUS (HIV)

(76) Inventor: Chih-Hui Lin, Taipei City (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 11/734,944

(22) Filed: Apr. 13, 2007

(51) Int. Cl. A61K 36/355; A61K 36/536; A61K 36/539; A61K 36/748; A61K 36/81; A61P 31/18

(52) U.S. Cl. 424/741; 424/725

(57) ABSTRACT

The invention provides a herbal composition for the inhibition of HIV, comprising an organic extract of Hedyotis diffusa (Oldenlandia diffusa), an organic solvent extract of Scutellaria barbata or Scutellaria rivularis, an organic solvent extract of Lonicera japonica or Lonicera confusato, an organic solvent extract of Prunella vulgari and an organi solvent extract of Solanum nigrum.

### FIELD OF THE INVENTION

This invention relates to herbal compositions comprising the extracts of herbs and their use in the inhibition of human immunodeficiency virus (HIV).

### BACKGROUND OF THE INVENTION

HIV (formally known as HTLV-III and lymphadenopathy-associated virus) is a retrovirus that is the cause of the disease known as AIDS (Acquired Immunodeficiency Syndrome), a syndrome where the immune system begins to fail, leading to many life-threatening opportunistic infections. HIV has been implicated as the primary cause of AIDS and can be transmitted via exposure to bodily fluids. In addition to percutaneous injury, contact with mucous membranes or non-intact skin with blood, fluids containing blood, tissue or other potentially infectious bodily fluids pose an infectious risk. Infection of human CD-4+T-lymphocytes with an HIV virus leads to depletion of this cell population, resulting in an immunodeficient state, and eventually opportunistic infections, neurological dysfunctions, neoplastic growth, and ultimately death. There are currently 18 drugs licensed and used for the treatment of HIV, and these drugs are divided into one of four classes depending on how they attack HIV. Drugs in the class of nucleoside/nucleotide reverse transcriptase inhibitors are AZT (zidovudine, Retrovir), ddI (didanosine, Videx), 3TC (lamivudine, Epivir), d4T (stavudine, Zerit), abacavir (Ziagen), and FTC (emtricitabine, Emtriva). Drugs in the class of non-nucleoside reverse transcriptase inhibitors are efavirenz (Sustiva) and nevirapine (Viramune). Drugs in the class of protease inhibitors are lopinavir/ritonavir (Kaletra), indinavir (Crixivan), ritonavir (Norvir), nelfinavir (Viracept), saquinavir hard gel capsules (Invirase), atazanavir (Reyataz), amprenavir (Agenerase), fosamprenavir (Telzir), and tipranavir (Aptivus). Only one drug is available in the class of fusion inhibitor, T20 (enfuvirtide, Fuzeon). The antiretroviral drugs are usually combined into three-drug cocktails called highly active antiretroviral therapy or HAART. However, the above-mentioned drugs still cannot effectively treat AIDS.

U.S. Pat. No. 5,837,257 provides a method for treating a subject having a viral infection selected from the group consisting of hepatitis B virus (HBV), hepatitis C virus (HCV), leukemia virus (LV) and human immunodeficiency virus (HIV), comprising administering to said subject in need thereof a therapeutically effective amount of a composition comprising a) SOLANI HERBA, prepared from the whole plant of Solanum nigrum; b) LESPEDEZAE HERBA, prepared from the whole plant of Lespedeza cuneata; c) SENECINIS HERBA, prepared from the whole plant of Senecio scandens; and d) LIGUSTRI FRUCTUS. The herbal materials of U.S. Pat. No. 5,837,257 were extracted with boiling water on a basis of 1 part by weight of plant material to approximately 5 to 10 parts by weight of water. However, the water extracts disclosed in U.S. Pat. No. 5,837,257, alone or in combination, do not contain effective anti-viral components and thus are not effective in the inhibition of HIV.

### SUMMARY OF THE INVENTION

The invention provides a herbal composition for the inhibition of HIV, comprising:
a) an organic solvent extract of Hedyotis diffusa (Oldenlandia diffusa);
b) an organic solvent extract of Scutellaria barbata or Scutellaria rivularis;
c) an organic solvent extract of Lonicera japonica or Lonicera confusato;
d) an organic solvent extract of Prunella vulgaris; and
e) an organic solvent extract of Solanum nigrum.
wherein the said composition comprises more than 0.7% by weight of chlorogenic acid and more than 0.2% by weight of ursolic acid.

### DESCRIPTION OF THE INVENTION

The invention relates to an herbal composition for the inhibition of HIV. The present invention discovers an antiviral herbal composition produced by extracting several herbs with an organic solvent. The composition of the invention can effectively inhibit HIV without toxicity.

The invention provides a mixture for the inhibition of HIV, comprising:
a) an organic solvent extract of Hedyotis diffusa (Oldenlandia diffusa);
b) an organic solvent extract of Scutellaria barbata or Scutellaria rivularis;
c) an organic solvent extract of Lonicera japonica or Lonicera confusato;
d) an organic solvent extract of Prunella vulgaris; and
e) an organic solvent extract of Solanum nigrum;
wherein the said composition comprises more than 0.7% by weight of chlorogenic acid and more than 0.2% by weight of ursolic acid.

According to the invention, the organic solvent used to extract the herbs of the mixture of the invention is selected from the group consisting of alcohol, ketone, hexane or chloroform. Preferably, the organic solvent is an alcohol selected from the group consisting of methanol, ethanol, propanol and isopropanol. More Preferably, the organic solvent is acetone.

According to the invention, the amounts of the extracts of a), b), c), d) and e) in the herbal composition of the invention range from 10-30 wt %, 10-30 wt %, 10-30 wt %, 10-30 wt % and 10-30 wt %, respectively. Preferably, the amounts of the extracts of a), b), c), d) and e) in the herbal composition of the invention range from 15-25 wt %, 15-25 wt %, 15-25 wt %, 15-25 wt % and 15-25 wt %, respectively. More Preferably, the amounts of the extracts of a), b), c), d) and e) in the herbal composition of the invention are about 20 wt %, about 20 wt %, about 20 wt %, about 20 wt % and about 20 wt %.

A herbal medicine known as Hedyotis Diffusa (Synonyms: Oldenlandia diffusa; common name: Snake-needle grass), has traditionally been used to treat illnesses such as malignant swelling, urethra infection, pharyngitis, laryngitis, tonsillitis, toxic snake bites, sub-acute or chronic coccygodynia, prurigo, carbuncle, appendicitis, intestinal cancer, contusion injuries and eye diseases; also, it reduces inflammation, relieves pain and is diuretic and antibacterial. It acts mainly on the liver and also stimulates the immune system. Hedyotis diffusa (Oldenlandia diffusa) belongs to the family Rubiaceae, which tastes sweet and is nontoxic. According to the invention, the whole plant of Hedyotis diffusa (Oldenlandia diffusa) is used to prepare the herbal compositions of the invention.

A Chinese herbal medicine known as Scutellariae Barbatae has traditionally been used to treat illnesses such as hematemesis, gonorrhea with traces of blood, jaundice, sore throats, lung abscesses, boils, carbuncles, abscesses, neck lymph node swelling, sores, cancer, contusion or cut injuries, snake bite injuries, dysentery with traces of blood, convulsions, pneumonia, abdominal pains, congenital diseases, enteritis, coccygodynia, appendicitis, asthma, malaria, and rheumatism. Scutellaria barbata is a plant used in traditional Chinese medicine. Other names include ban zhi lian (Chinese), scullcap, barbat skullcap, skute barbata, or herba scutellariae barbatae. It was also found to have an antibacterial effect. Scutellariae Barbatae is prepared from the dried whole plant of Scutellaria barbata (Scutellaria rivularis) or Scutellaria dependens which belong to the family Labiatae. According to the invention, the whole plant of Scutellaria barbata or Scutellaria rivularis is used in the preparation of the herbal compositions of the invention.

A Chinese herbal medicine known as Lonicerae Flos (common name: Japanese honeysuckle) has traditionally been used to treat illnesses such as fever, febrile diseases, acute infectious diseases, measles, carbuncle, dysentery, malignant sores and swelling, abscesses, boils, gonorrhea, syphilis, poisoning, enteritis, swelling, ringworm and similar skin diseases. The stems are used internally in the treatment of acute rheumatoid arthritis, mumps and hepatitis. The herbal medicine tastes sweet. The anti-HIV activity in vitro of a mixture of Lonicera japonica, Isatis tinctoria (or Isatis indigotica) and Polygonum bistorta or a mixture of Lonicera japonica with Scutellaria baicalensis is known. Water extractions of the mixtures, treatment with ethanol for precipitation and charcoal adsorption are disclosed for the preparation for the anti-HIV active composition (U.S. Pat. No. 5,178,865). According to the invention, the flower of Lonicera japonica or Lonicera confusato is used in the preparation of the herbal compositions of the invention.

A Chinese herbal medicine known as Prunellae Spica (common name: Self-heal) has traditionally been used to treat antibacterial, antipyretic, antiseptic, antispasmodic, astringent, carminative, diuretic, febrifuge, hypotensive, stomachic, and styptic illnesses, as well as goiter, scrofula, neck lymph node tuberculosis, lymph node swelling, eye redness, pain, abscesses, sores, hemorrhoids, swollen eye, ophthalmalgia, leucorrhoea with traces of blood, gonorrhea, uterine disease, mastitis, breast abscesses, breast cancer, foot swelling, paralysis, chronic arthritis, conjunctivitis, and hypertension. Prunellae Spica is prepared from the dried spica or whole plant of Prunella vulgaris or Prunella vulgaris subsp. Both plants belong to the family Labiatae. The whole plant can be used as a diuretic and also has antibacterial effects in vitro. The herbal medicine tastes bitter and is nontoxic. According to the invention, the whole plant of Prunella vulgaris is used in the preparation of the herbal compositions of the invention.

A Chinese herbal medicine known as Solanum Nigrum L. (common name: Black nightshade) has traditionally been used (the whole plant) as an antiperiodic, antiphlogistic, diaphoretic, diuretic, emollient, febrifuge, narcotic, purgative and sedative. The plant has been used in the manufacture of locally analgesic ointments and the juice of the fruit has been used as an analgesic for toothaches. According to the invention, the whole plant of Solanum nigrum is used in the preparation of the herbal compositions of the invention.

According to the invention, it is found that Hedyotis diffusa and Prunella vulgari do not have effect in inhibiting HIV-1 reverse transcriptase. The herbal composition comprises chlorogenic acid and ursolic acid in significant amounts. Both chlorogenic acid and ursolic acid can be used as markers of HPLC to trace the preparation of the herbal composition of the invention. Chlorogenic acid can be used as an anti-infectious active ingredient, it has wide anti-viral, anti-bacterial effects, and has relatively lower toxicity and side effects. Similarly, ursolic acid is known in the inhibition of viruses. The herbal composition of the invention has chlorogenic acid and ursolic acid, in particular, in the amounts of more than 0.25%-1% by weight and more than 0.1%-0.5% by weight, respectively.

The herbal composition of the invention can be prepared by extracting each herb with an organic solvent and then mixing the resulting extracts. Alternatively, the herbal composition of the invention can be prepared by mixing all herbs and then extracting them with an organic solvent. The extraction can use various methods known in the art. A suitable part or parts or the whole herb of an herb plant(s) were obtained and washed with cold water. The plant materials were soaking in organic solvent for over 24 hours. One part by weight of plant material(s) to approximately 5 to 10 parts by weight of organic solvent was used as an extraction ratio. The extraction solution was separated from the plant material by filtration. Subsequently, the resulting solution was concentrated, and the concentrate may then be spray dried or freeze dried or absorbed by powdered material of the same plant material or starch and thus, the herbal medicine is prepared in powdered form.

In preferred embodiments, the composition of this invention further contains a pharmaceutically acceptable carrier. As used herein, the term “pharmaceutically acceptable carrier” is meant to include one or more pharmaceutically suitable, inactive excipients, carriers, diluents, adjuvants, and lubricants. Non-limiting examples of inactive excipients, carriers, diluents, lubricants, and adjuvants which can be used in the composition of the present invention include: cellulose, substituted cellulose, calcium carbonate, dicalcium phosphate, starches, lactose, modified food starches, dextrose, calcium sulfate, magnesium carbonate, magnesium stearate, stearic acid, glycerin, vegetable oils, polysorbates, lecithin, silicium dioxide, food glaze, talc, croscarmeilose sodium, povidone, water and gelatin. Additional inactive excipients, carriers, diluents, lubricants and adjuvants which may be used with the active-ingredient composition of this invention are known in the art. The herbal compositions of the invention can be formulated into preparations in the form of a solution, tablet, capsule, powder, candy, gel or emulsion. The herbal compositions of the invention can be prepared as nutritional products or pharmaceutical products.

The composition of the invention may be formulated into enteral and parenteral preparations containing an amount of the composition of the invention that is effective for inhibiting HIV. The compositions of the invention can be administered in a therapeutically effective amount of 3 to 30 grams per day. According to the testing data provided by the Development Center for Biotechnology (DCB, Taipei County, Taiwan), the compositions of the invention have no side effects in 5,000 mg/Kg in rats. The dosage of the composition of the invention may range from 3 g to 30 g per day and the composition is administered at least three (3) times per day. Preferably, the dosage of the composition of the invention ranges from 15 g to 27 g per day. It has been found that the composition of the invention is effective in reducing HIV load in carriers. The herbal composition of the invention has been found effective in treating HIV-infected patient. It was found that a dosage as high as 30 gm per day does not have side effects.

### EXAMPLE

### Example 1

### Cytotoxicity Assay of Anti-Human Immunodeficiency Virus

The Hedyotis diffusa, Scutellaria barbata, Lonicera japonica, Prunella vulgaris and Solanum nigrum were extracted with ethanol, respectively. The herbal composition of the invention was prepared by mixing the extracts of the above-mentioned herbs with the ratio of 1:1:1:1:1 by weight. The resulting mixture was prepared as powders, respectively.

The cytotoxicity assay was conducted in the Children's Hospital of Philadelphia (34^{th} Street and Civic Cancer Boulevard, Philadelphia, Pa. 19104-4399). Different HIV strains (R5 strain Bal, R5 X4 strain 89.6, X4 strain UGO24) were used in the assay. The chronically HIV infected human T-lymphocyte cell line (ACH-2) was used to test the anti-HIV effects of the herbal compositions of the invention. ACH-2 cells were treated with the herbal compositions of the invention in different concentrations for 72 hours. The treated cells were stained with Trypan Blue and counted under a microscope. 55% of ACH-2 cells were dead after being treated with the herbal compositions of the invention. In addition, a chronically HIV-infected human monocytic cell line (U1) was used in the assays. U1 cells were treated with different concentrations of the herbal composition of the invention for 96 hours. The treated cells were stained with Trypan Blue and counted under a microscope. 50% of U1 cells were dead after being treated with the herbal compositions of the invention.

### Example 2

### Acute Oral Toxicity Test of HIV in Rats

An acute oral toxicity test of the herbal compositions of the invention was conducted in Sprague-Dawley (SD) rats to detect the toxicity of the herbal composition of the invention. 96 SD rats were divided into four groups. Each group included six male and six female rats. One group was administered with water (injection grade) as control and the other three groups were administered with the herbal compositions of the invention at doses of 1000, 3000 and 5000 mg/kg via oral gavage. The rats were observed for 14 days. No mortality was observed in both the control and test groups during the 14-day study period. There was no difference in the body weights between the control group and test groups. No gross lesions were observed in the rats of both the test and control groups. This study suggests that the herbal compositions of the invention cause no significant toxicity in the SD rats at a dose of up to 5000 mg/kg.

### Example 3

### 27-Day Subacute Oral Toxicity Test in Rats

The herbal compositions of the invention were tested in SD rats to determine the numbers of Observed Adverse Effect Level (NOAEL). Four groups of Sprague-Dawley (SD) rats, each including 6 male and 6 female rats, were administered daily via oral gavage for 27 days. The herbal compositions of the invention were prepared as a solution with concentrations of 0 mg/ml, 200 mg/ml, 400 mg/ml and 500 mg/ml. The administration doses were 0 mg/kg (control group), 2000 mg/kg (low dosage group), 4000 mg/kg (medium dosage group) and 5000 mg/kg (high dosage group). The parameters, general demeanor, clinical signs, mortality, body weights/total body weight gains and histopathological changes in liver and kidneys were examined. There were no significant differences between the rats of the control, low-dosage, medium-dosage and high-dosage groups in all parameters. In addition, no treatment-related histopathological change was observed. In conclusion, the results of this study indicated that SD rats which ingested the herbal composition of the invention solution at doses of up to 5000 mg/kg/day for 27 days did not exhibit any significant adverse effects. Therefore, the 27-day oral NOAEL for rats is greater than 5000 mg/kg/day. The doses (2000, 4000 and 5000 mg/kg/day) of the herbal compositions of the invention tested in this study provide 6.7, 13.3, and 16.7 fold safety margins over the anticipated recommended human dosage and the corresponding safety margins of 1, 2.1 and 2.7 were reached based on the conversion of surface area.

### Example 4

### 90-Day Subchronic Oral Toxicity Test in Rats

A 90-day sub-chronic oral toxicity test in rats was conducted to evaluate the toxicity of the herbal composition of the invention and to determine “maximum tolerated dose” (MTD), when administered to Sprague-Dawley (SD) rats daily for 90 days via oral gavage. The rats in four groups, each group including 12 male and 12 female rats, were dosed daily via oral gavage for 90 consecutive days. An additional 6 male and 6 female rats were treated for 90 days and withdrawn from treatment for 28 days as recovery groups. The herbal compositions of the invention were prepared as solutions at concentrations of 0 mg/ml, 50 mg/ml, 150 mg/ml and 500 mg/ml. The doses were 0 mg/kg/day (injection-grade water), 500 mg/kg/day, 1500 mg/kg/day and 5000 mg/kg/day. The results of this study indicated that SD rats treated with the herbal compositions of the invention at a dose of up to 5000 mg/kg/day for 90 consecutive days did not exhibit any significant adverse effects.

Patients were instructed to take the herbal compositions of the invention three (3) times a day, 5-9 g each time. Unit dosages of the herbal compositions of the invention were prepared in capsule. Each unit dose (500 mg) of the herbal composition of the invention was mixed with warm water and taken orally.

### Example 5

### Clinical Trial

The herbal composition of the invention comprising five herbal medicines as claimed herein was manufactured following good manufacture practice (GMP) guidelines.

A clinical trial using the herbal composition of the invention was conducted in four patients and its results are in the tables below:

| Patient | | Sampling | CD4 | Virus load |
|---|---|---|---|---|
| No. | Sex (Male/Female, M/F) | Time | M1 | copies |
| 103 | M | screening | 810 | 75600 |
| 103 | M | Week 4 | 615 | 5840 |
| 103 | M | Week 8 | 522 | 13300 |
| 103 | M | Week 12 | 569 | 4010 |
| 103 | M | Week 16 | 549 | 8760 |
| 103 | M | Week 20 | 597 | 28100 |
| 103 | M | Week 24 | 486 | 2010 |

| Patient | | Sampling | CD4 | Virus load |
|---|---|---|---|---|
| No. | Sex (Male/Female, M/F) | Time | M1 | copies |
| 111 | M | Day 1 | 660 | 73800 |
| 111 | M | Week 8 | 563 | 44100 |
| 111 | M | Week 12 | 659 | 54500 |
| 111 | M | Week 16 | 543 | 33300 |
| 111 | M | Week 20 | 547 | 31100 |
| 111 | M | Week 24 | 617 | 16500 |
| 111 | M | Month 7 | 329 | 34200 |

| Patient | | Sampling | CD4 | Virus load |
|---|---|---|---|---|
| No. | Sex (Male/Female, M/F) | Time | M1 | copies |
| 147 | M | Day 1 | 327 | 53900 |
| 147 | M | Week 4 | 244 | 154000 |
| 147 | M | Week 8 | 231 | 62700 |
| 147 | M | Week 12 | 225 | 255000 |
| 147 | M | Week 16 | 271 | 238000 |
| 147 | M | Week 24 | 230 | 340000 |
| 147 | M | Month 7 | 230 | 197000 |

| Patient | | Sampling | CD4 | Virus load |
|---|---|---|---|---|
| No. | Sex (Male/Female, M/F) | Time | M1 | copies |
| 157 | M | Day 1 | 264 | 7590 |
| 157 | M | Week 4 | 271 | 13700 |
| 157 | M | Week 8 | 270 | 7880 |
| 157 | M | Week 12 | 247 | 6480 |
| 157 | M | Week 24 | 348 | 709 |

The above results clearly show that after two months, the herbal composition of the invention can significantly reduce the HIV virus and is effective in treating HIV infected patients. It is therefore an aspect of this invention that the antiviral herbal medicines including the herbal composition according to this invention at various proportions and effective doses are effective in treating HIV infection, i.e., AIDS.

The medical community is constantly in search of methods and products that will effectively treat viral infections, especially methods and products for treating humans infected with HIV. While certain representative embodiments have been described herein, it will be apparent to those skilled in the art that various changes and modifications may be made therein without departing from the spirit or scope of this invention.

What is claimed is:

1. A herbal composition having a cytotoxic effect on HIV infected cells, comprising:
a) an organic solvent extract of Hedyotis diffusa (Oldenlandia diffusa);
b) an organic solvent extract of Scutellaria barbata or Scutellaria rivularis;
c) an organic solvent extract of Lonicera japonica or Lonicera confusato;
d) an organic solvent extract of Prunella vulgaris; and
e) an organic solvent extract of Solanum nigrum;
wherein more than 0.7% by weight of chlorogenic acid and more than 0.2% by weight of ursolic acid.

2. The herbal composition of claim 1, wherein the organic solvent is alcohol, ketone, hexane or chloroform.

3. The herbal composition of claim 1, wherein the alcohol is selected from the group consisting of methanol, ethanol, propanol and isopropanol.

4. The herbal composition of claim 1, wherein the ketone is acetone.

5. The herbal composition of claim 1, wherein the amounts of chlorogenic acid and ursolic acid range from 0.25%-1% by weight and more than 0.1%-0.5% by weight, respectively.

6. The herbal composition of claim 1, which has a therapeutically effective amount of 3 to 30 grams per day.

7. The herbal composition of claim 1, which has a therapeutically effective amount of 15 g to 27 g per day.

8. The herbal composition of claim 1, wherein the amounts of the extracts of a), b), c), d) and e) range from 10-30 wt %, 10-30 wt %, 10-30 wt %, 10-30 wt % and 10-30 wt %, respectively.

9. The herbal composition of claim 1, wherein the amounts of the extracts of a), b), c), d) and e) range from 15-25 wt %, 15-25 wt %, 15-25 wt %, 15-25 wt % and 15-25 wt %, respectively.

10. The herbal composition of claim 1, wherein the amounts of the extracts of a), b), c), d) and e) are about 20 wt %, about 20 wt %, about 20 wt %, about 20 wt % and about 20 wt %

11. The herbal composition of claim 1, which can be further formulated into preparations in the form of a solution, tablet, capsule, powder, candy, gel or emulsion.
